SEQUENCE LISTING (2 sequences)

SEQ ID NO: 1 - DNA, 34 bp, Artificial Sequence
  Description of Artificial Sequence Synthetic primer
agcgctagaccatgtattcctaagtccttcggtt

SEQ ID NO: 2 - DNA, 36 bp, Artificial Sequence
  Description of Artificial Sequence Synthetic primer
ggtaccttattgtctgtgccacaagtaggtgtggat

(19) United States
(12) Patent Application Publication
(10) Pub. No.: US 2005/0265988 A1
(43) Pub. Date: Dec. 1, 2005

(54) Glycosylated glucocerebrosidase expression in fungal hosts

(76) Inventors: Byung-Kwon Choi, Norwich, VT (US); Sandra Edith Rios, Lebanon, NH (US); Stefan Wildt, Lebanon, NH (US); Tillman Ulf Gerngross, Hanover, NH (US)

Correspondence Address: Chang B. Hong, Suite 200, 21 Lafayette St., Lebanon, NH, 03766, US

(21) Appl. No.: 11/083,446

(22) Filed: Mar. 18, 2005

Related U.S. Application Data
(60) Provisional application No. 60/554,522, filed on Mar. 18, 2004.

(51) Int. Cl. A61K 38/47; C07H 21/04; C12P 21/06; C12N 9/24; C12N 1/18; C12N 15/74

(52) U.S. Cl. 424/94.61; 435/69.1; 435/254.2; 435/483; 435/200; 536/23.2

(57) ABSTRACT

A recombinant fungal host cell producing recombinant glucocerebrosidase is provided. A functional recombinant glucocerebrosidase produced in recombinant fungal host cells is also provided. Methods for producing and isolating functional recombinant glucocerebrosidase from fungal hosts are also provided.

### CROSS-REFERENCE TO RELATED APPLICATION

This application claims priority to U.S. provisional application Ser. No. 60/554,522, Mar. 18, 2004, which is incorporated by reference herein in its entirety.

### FIELD OF THE INVENTION

The present invention generally relates to compositions and methods for producing therapeutic proteins in lower eukaryotes. The present invention more specifically relates to novel fungal host cells producing glucocerebrosidase and glucocerebrosidase compositions comprising terminal mannose residues on an N-linked glycan.

### BACKGROUND OF THE INVENTION

Gaucher's disease is the most common lysosomal storage disorder. Deficient activity of β-glucocerebrosidase (EC 3.2.1.45) caused by mutations in the relevant gene, results in the appearance of abnormal macrophages, known as Gaucher's cells. β-gluco-cerebrosidase is a lysosomal hydrolase participating in the breakdown of membrane glycosphingolipids. Specifically, glucocerebrosidase is required for hydrolysis of glucocerebroside to glucose and cerebroside. As there are no alternative pathways, a deficient β-glucocerebrosidase results in the accumulation of glucocerebroside glycolipid predominantly in tissue macrophages (Friedman, et al., Blood, 93, 2807-2816, 1999). These lipid-laden macrophages are present in the liver, spleen, bone and lungs of Gaucher's patients. Northern blot analysis of Gaucher patients revealed that the glucocerebrosidase transcript is normal, whereas Western analysis showed a lack of the processed 56 kD isoform of the enzyme (Park et al., Pediatr Res 53, 387-395, 2003). Enzyme replacement therapy has been successful in alleviating many of the symptoms associated with non-neuronopathic, type 1 Gaucher disease.

The current enzyme therapy is primarily recombinant glucocerebrosidase expressed in Chinese Hamster Ovary (CHO) cells or derived from human placentae (Barton et al., N Eng. J Med 324, 1464-1470, 1991; Grabowski et al., Ann Intern Med 122, 33-39, 1995). Glycoproteins from either CHO cells or human placentae receive complex N-glycan modifications-N-acetylglucosamine (GlcNAc), galactose (Gal) and terminal sialic acid (NANA). However, a more therapeutically active form of glucocerebrosidase having terminal mannose groups (e.g., Man₃GlcNAc₂(Fuc)—Man₉GlcNAc₂(Fuc)) which are responsible for selective delivery of this protein to macrophages (Friedman, et al., Blood, 93, 2807-2816, 1999). It has therefore been necessary to enzymatically remove the complex glycans after isolation of the glycoprotein from these mammalian host cells. To do so, in vitro treatment with neuraminidase (to remove sialic acid), galactosidase (to remove galactose) and hexosaminidase (to remove N-acetylglucosamine) are required. The reactions inherently result in incomplete conversion of these glycans to the preferred terminal mannose groups, and the incompletely converted glycans are additionally subject to further mannosylation, resulting in a heterogenous pool of proteins. Further, while it is known that glucocerebrosidase with terminal mannose groups is more therapeutically active, it is not known whether this activity can be improved with either a homogenous pool or a specific heterogenous pool of mannose groups.

Using currently available processes with mammalian host cells, the heterogenous mixture of glycans ranging from Man₃GlcNAc₂(Fuc) to Man₉GlcNAc₂Fuc cannot be precisely controlled—i.e. glycoprotein with specific mannose groups cannot be isolated. For example, the glycan profile of Cerezyme™ (FIG. 1) discloses a range of glycosylation structures. It is not known which particular N-glycan structure is best suited for the protein. Furthermore, the post-production processing in mammalian cells is a laborious and costly method, as opposed to a one-step or two-step isolation of glucocerebrosidase with terminal mannose from a lower eukaryotic host.

It would be useful to tailor the glucocerebrosidase protein to a particular glycan structure by modifying the glycosylation pattern on the protein. Accordingly, it is desirable to have a fungal-based expression system which can be engineered to produce glucocerebrosidase protein having a particular N-linked glycan comprising terminal mannose residues.

### SUMMARY OF THE INVENTION

The present invention provides methods for producing in a fungal host cell a glucocerebrosidase protein composition comprising Man₃GlcNAc₂, Man₅GlcNAc₂ and Man₈GlcNAc₂ glycans in homogenous pools or in combination in heterogenous pools. The invention also provides a method for the production of glucocerebrosidase protein composition with the desired Man₃GlcNAc₂, Man₅GlcNAc₂ and/or Man₈GlcNAc₂ glycans produced in vivo or in vitro. The invention further provides a method for the production of glucocerebrosidase protein with the desired Man₃GlcNAc₂, Man₅GlcNAc₂ and/or Man₈GlcNAc₂ glycans in the yeast, Pichia pastoris.

It has been observed that enzyme replacement treatment of lysosomal storage diseases, and Gaucher Disease in particular, requires not only sufficient expression of recombinant protein, but also that the recombinant protein sufficiently find its way into specific cells, particularly cells of the liver, such as hepatocytes and macrophages (e.g., Kupfer cells), in order to have the desired effect. See, Beck, Expert Opin. Investig. Drugs (2002) 11(6):851-858. Thus, while not being bound by any particular theory, the present inventors hypothesized that, in order to optimize the effectiveness of enzyme replacement therapy in the treatment of lysosomal storage diseases, it would be desirable to produce such recombinant lysosomal enzymes, such as glucocerebrosidase, with specifically directed glycosylation patterns. In this manner, the recombinant lysosomal enzymes could be specifically directed to bind to specific cellular receptors, but not others. In particular, the inventors hypothesized that the production of recombinant glucocerebrosidase which is essentially free of high-mannose, could more effectively bind to specific mannose receptors, and might be more efficiently taken up by the cells needed for the processing of glycolipids involved in lysosomal storage diseases. The inventors further hypothesized that the present invention would have other potential advantages by virtue of providing for more homogeneous forms of recombinant glucocerebrosidase protein compositions, with mannose residues having lower mass or density than that of existing glucocerebrosidase treatments. Further, because the present invention produces recombinant glucocerebrosidase protein compositions which have a particular glycosylation pattern comprising terminal mannose residues, but which are essentially free of fucose and galactose, the compositions of the present invention may have increased activity and potency without provoking an adverse immune response.

In certain embodiments, the present invention comprises compositions of recombinant glucocerebrosidase [rGCB] protein comprising an N-glycan structure that comprises predominantly a glycoform selected from the group consisting of: (a) Man₃GlcNAc₂, (b) Man₅GlcNAc₂, (c) Man₈GlcNAc₂ and a heterogenous pool of glycoforms (a) through (c) above. Preferably such compositions comprise less than 30 mole percent of N-glycans having a glycoform other than glycoforms (a) through (c). More preferably, the compositions comprise less than 20 mole percent of N-glycans having a glycoform other than glycoforms (a) through (c). Most preferably, the compositions comprise less than 10 mole percent of N-glycans having a glycoform other than glycoforms (a) through (c).

In other embodiments, the present invention provides compositions of recombinant glucocerebrosidase [rGCB] protein comprising an N-glycan structure that comprises predominantly a glycoform selected from the group consisting of (a) Man₃GlcNAc₂, (b) Man₅GlcNAc₂, (c) Man₈GlcNAc₂ glycans, or a heterogenous pool of glycoforms (a) through (c), wherein said composition of rGCB protein comprises at least 50 mole percent of glycoforms (a) through (c). It is preferred that said compositions comprise at least 60 mole percent of N-glycans having one of glycoforms (a) through (c). Most preferably, the compositions of the invention comprise at least 70 mole percent of N-glycans having one of glycoforms (a) through (c).

In yet other embodiments, the compositions of rGCB protein of the present invention comprise an N-glycan structure that comprises predominantly a glycoform selected from the group consisting of (a) Man₃GlcNAc₂, (b) Man₅GlcNAc₂, and/or (c) Man₈GlcNAc₂ glycans, wherein said composition of rGCB protein comprises at least 30 mole percent, preferably at least 40 mole percent; and most preferably at least 50 mole percent of a predominant N-glycan structure having fewer than 9 mannose residues.

In other preferred embodiments, the compositions of RGCB protein of the present invention comprise less than 30 mole percent high-mannose glycans [i.e., glycans having 9 or more mannose residues]. It is more preferred that said composition of rGCB protein comprise less than 20 mole percent high-mannose glycans; and most preferably, said compositions of rGCB protein comprises less than 10 mole percent high-mannose glycans.

In yet other preferred embodiments, the composition of the present invention comprise rGCB protein and is essentially free of fucose and/or galactose residues.

The present invention also provides methods for producing a composition of recombinant glucocerebrosidase [rGCB] protein in a lower eukaryotic host cell. Said lower eukaryotic host cell is typically lacking at least one functional enzyme involved in hypermannosylation of proteins. The methods of the present invention comprise:
a. transforming said lower eukaryotic host cell with a recombinant nucleotide sequence encoding an rGCB protein; and
b. culturing said lower eukaryotic host cell in conditions essentially free of neuraminidase or galactosidase suitable for expression of the rGCB protein to produce predominantly a glycoform selected from the group consisting of (a) Man₃GlcNAc₂, (b) Man₅GlcNAc₂, (c) Man₈GlcNAc₂ glycans, or a heterogenous pool of glycoforms (a) through (c).

In the methods of the present invention, it is preferred that the lower eukaryotic host cell lacks at least one functional enzyme involved in hypermannosylation selected from the group consisting of: the ALG3 gene; and the OCH1 gene. In other embodiments, it is preferred that the lower eukaryotic host cell expresses at least one exogenous gene selected from the group consisting of mannosidases; mannosyltransferases; N-acetylglucosaminyltransferases; UDP-N-acetylglucosamine transporters; and phosphomannosyltransferases.

In the methods of the present invention it is further preferred that the lower eukaryotic cell is selected from the following species: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

In other preferred embodiments of the methods of the present invention, less than 30 mole percent of the N-glycans in said rGCB protein composition comprises high-mannose glycans. More preferably, less than 20 mole percent of the N-glycans in said rGCB protein composition comprises high-mannose glycans. And most preferably, less than 10 mole percent of the N-glycans in said rGCB protein composition comprises high-mannose glycans.

In still other preferred embodiments, the methods of the present invention produce rGCB protein compositions wherein less than 30 mole percent of the N-glycans in said rGCB protein composition comprises a glycan other than (a) Man₃GlcNAc₂, (b) Man₅GlcNAc₂, or (c) Man₈GlcNAc₂. More preferably, less than 20 mole percent of the N-glycans in said rGCB protein composition comprises a glycan other than (a) Man₃GlcNAc₂, (b) Man₅GlcNAc₂, or (c) Man₈GlcNAc₂. And most preferably, less than 10 mole percent of the N-glycans in said rGCB protein composition comprises a glycan other than (a) Man₃GlcNAc₂, (b) Man₅GlcNAc₂, or (c) Man₈GlcNAc₂.

In still other embodiments, the method of the present invention results in the production of rGCB protein compositions, wherein at least 50 mole percent of the N-glycans in said rGCB protein composition comprises Man₃GlcNAc₂, Man₅GlcNAc₂, or Man₈GlcNAc₂. It is preferred that at least 60 mole percent of the N-glycans in said rGCB protein composition comprises Man₃GlcNAc₂, Man₅GlcNAc₂, or Man₈GlcNAc₂. In more preferred embodiments, at least 70 mole percent of the N-glycans in said rGCB protein composition comprises Man₃GlcNAc₂, Man₅GlcNAc₂, or Man₈GlcNAc₂.

It is preferred in the methods of the present invention that at least 30 mole percent, more preferably at least 40 mole percent, and most preferably at least 50 mole percent of the N-glycans in said rGCB protein composition comprises a predominant N-glycan structure having fewer than 9 mannose residues.

In certain preferred embodiments of the present invention, the methods of the present invention produce compositions of rGCB protein essentially free of fucose. In other embodiments, the methods of the present invention produce compositions of RGCB protein that are essentially free of galactose.

The present invention also provides methods of treating patients having a Gaucher's type disease, comprising administration of a therapeutically effective amount of a recombinant glucocerebrosidase [rGCB] composition, said rGCB comprising predominantly N-glycan structures selected from the group consisting of (a) Man₃GlcNAc₂, (b) Man₅GlcNAc₂, (c) Man₈GlcNAc₂ glycoforms, and a heterogenous pool of glycoforms (a) through (c). It is preferred that said compositions of RGCB protein comprise at least 30 mole percent, preferably at least 40 mole percent, most preferably, at least 50 mole percent, of glycoforms (a) through (c). The rGCB compositions may comprise additional active agents, and may further comprise a pharmaceutically acceptable carrier.

Other rGCB protein compositions of the present invention may comprise an N-glycan structure in which at least 50 mole percent of the rGCB protein comprises 3 N-linked sites bearing predominantly a single glycoform selected from the group consisting of (a) Man₃GlcNAc₂, (b) Man₅GlcNAc₂ and (c) Man₈GlcNAc₂ glycans. In more preferred embodiments, at least 60 mole percent, or most preferably at least 70 mole percent of the rGCB protein comprises 3 N-linked sites bearing predominantly a single glycoform selected from the group consisting of (a) Man₃GlcNAc₂, (b) Man₅GlcNAc₂ and (c) Man₈GlcNAc₂ glycans.

### BRIEF DESCRIPTION OF THE DRAWINGS

FIG. 1. depicts a positive-ion MALDI-TOF MS of N-linked glycans of glucocerebrosidase (Cerezyme™) produced from CHO cells.

FIG. 2. Positive-ion MALDI-TOF MS of N-linked glycans. Man₃GlcNAc₂ glycans from P. pastoris YSH44 treated with hexosaminidase (Panel A). Man₅ and Man₈ glycans from P. pastoris YJN168 (Panel B). Man₅GlcNAc₂ glycans from P. pastoris YJN188 (Panel C).

FIG. 3. Immunoblot of fractions of purified GCB produced in P. pastoris BK303 collected from Ni-affinity column using anti-His antibodies to illuminate peak fractions containing GCB-His.

### DETAILED DESCRIPTION OF THE INVENTION

Unless otherwise defined herein, scientific and technical terms used in connection with the present invention shall have the meanings that are commonly understood by those of ordinary skill in the art. Further, unless otherwise required by context, singular terms shall include the plural and plural terms shall include the singular. Generally, nomenclatures used in connection with, and techniques of biochemistry, enzymology, molecular and cellular biology, microbiology, genetics and protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al. Molecular Cloning: A Laboratory Manual, 2d ed., Cold Spring Harbor Laboratory Press, Cold Spring Harbor, N.Y. (1989); Ausubel et al., Current Protocols in Molecular Biology, Greene Publishing Associates (1992, and Supplements to 2002); Harlow and Lane, Antibodies: A Laboratory Manual, Cold Spring Harbor Laboratory Press, Cold Spring Harbor, N.Y. (1990); Taylor and Drickamer, Introduction to Glycobiology, Oxford Univ. Press (2003); Worthington Enzyme Manual, Worthington Biochemical Corp., Freehold, N.J.; Handbook of Biochemistry: Section A Proteins, Vol I, CRC Press (1976); Handbook of Biochemistry: Section A Proteins, Vol II, CRC Press (1976); Essentials of Glycobiology, Cold Spring Harbor Laboratory Press (1999).

All publications, patents and other references mentioned herein are hereby incorporated by reference in their entireties.

The following terms, unless otherwise indicated, shall be understood to have the following meanings:

The term “polynucleotide” or “nucleic acid molecule” refers to a polymeric form of nucleotides of at least 10 bases in length. The term includes DNA molecules (e.g., cDNA or genomic or synthetic DNA) and RNA molecules (e.g., mRNA or synthetic RNA), as well as analogs of DNA or RNA containing non-natural nucleotide analogs, non-native internucleoside bonds, or both. The nucleic acid can be in any topological conformation. For instance, the nucleic acid can be single-stranded, double-stranded, triple-stranded, quadruplexed, partially double-stranded, branched, hairpinned, circular, or in a padlocked conformation.

An “isolated” or “substantially pure” nucleic acid or polynucleotide (e.g., an RNA, DNA or a mixed polymer) is one which is substantially separated from other cellular components that naturally accompany the native polynucleotide in its natural host cell, e.g., ribosomes, polymerases and genomic sequences with which it is naturally associated. The term embraces a nucleic acid or polynucleotide that (1) has been removed from its naturally occurring environment, (2) is not associated with all or a portion of a polynucleotide in which the “isolated polynucleotide” is found in nature, (3) is operatively linked to a polynucleotide which it is not linked to in nature, or (4) does not occur in nature. The term “isolated” or “substantially pure” also can be used in reference to recombinant or cloned DNA isolates, chemically synthesized polynucleotide analogs, or polynucleotide analogs that are biologically synthesized by heterologous systems.

However, “isolated” does not necessarily require that the nucleic acid or polynucleotide so described has itself been physically removed from its native environment. For instance, an endogenous nucleic acid sequence in the genome of an organism is deemed “isolated” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “isolated” because it is separated from at least some of the sequences that naturally flank it.

A nucleic acid is also considered “isolated” if it contains any modifications that do not naturally occur to the corresponding nucleic acid in a genome. For instance, an endogenous coding sequence is considered “isolated” if it contains an insertion, deletion or a point mutation introduced artificially, e.g., by human intervention. An “isolated nucleic acid” also includes a nucleic acid integrated into a host cell chromosome at a heterologous site and a nucleic acid construct present as an episome. Moreover, an “isolated nucleic acid” can be substantially free of other cellular material, or substantially free of culture medium when produced by recombinant techniques, or substantially free of chemical precursors or other chemicals when chemically synthesized.

As used herein, the phrase “degenerate variant” of a reference nucleic acid sequence encompasses nucleic acid sequences that can be translated, according to the standard genetic code, to provide an amino acid sequence identical to that translated from the reference nucleic acid sequence. The term “degenerate oligonucleotide” or “degenerate primer” is used to signify an oligonucleotide capable of hybridizing with target nucleic acid sequences that are not necessarily identical in sequence but that are homologous to one another within one or more particular segments.

In general, “stringent hybridization” is performed at about 25° C. below the thermal melting point (Tₘ) for the specific DNA hybrid under a particular set of conditions. “Stringent washing” is performed at temperatures about 5° C. lower than the Tₘ for the specific DNA hybrid under a particular set of conditions. The Tₘ is the temperature at which 50% of the target sequence hybridizes to a perfectly matched probe. See Sambrook et al., Molecular Cloning: A Laboratory Manual, 2d ed., Cold Spring Harbor Laboratory Press, Cold Spring Harbor, N.Y. (1989), page 9.51, hereby incorporated by reference. For purposes herein, “stringent conditions” are defined for solution phase hybridization as aqueous hybridization (i.e., free of formamide) in 6×SSC (where 20×SSC contains 3.0 M NaCl and 0.3 M sodium citrate), 1% SDS at 65° C. for 8-12 hours, followed by two washes in 0.2×SSC, 0.1% SDS at 65° C. for 20 minutes. It will be appreciated by the skilled worker that hybridization at 65° C. will occur at different rates depending on a number of factors including the length and percent identity of the sequences which are hybridizing.

The nucleic acids (also referred to as polynucleotides) of this invention may include both sense and antisense strands of RNA, cDNA, genomic DNA, and synthetic forms and mixed polymers of the above. They may be modified chemically or biochemically or may contain non-natural or derivatized nucleotide bases, as will be readily appreciated by those of skill in the art. Such modifications include, for example, labels, methylation, substitution of one or more of the naturally occurring nucleotides with an analog, internucleotide modifications such as uncharged linkages (e.g., methyl phosphonates, phosphotriesters, phosphoramidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.) Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule. Other modifications can include, for example, analogs in which the ribose ring contains a bridging moiety or other structure such as the modifications found in “locked” nucleic acids.

The term “vector” as used herein is intended to refer to a nucleic acid molecule capable of transporting another nucleic acid to which it has been linked. One type of vector is a “plasmid”, which refers to a circular double stranded DNA loop into which additional DNA segments may be ligated. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell, and are thereby replicated along with the host genome. Moreover, certain preferred vectors are capable of directing the expression of genes to which they are operatively linked. Such vectors are referred to herein as “recombinant expression vectors” (or simply, “expression vectors”). Yeast vectors will often contain an origin of replication sequence from a 2 micron yeast plasmid, an autonomously replicating sequence (ARS), a promoter region, sequences for polyadenylation, sequences for transcription termination, and a selectable marker gene. Suitable promoter sequences for yeast vectors include, among others, promoters for metallothionein, 3-phosphoglycerate kinase (Hitzeman et al., J. Biol. Chem. 255:2073, (1980)) or other glycolytic enzymes (Holland et al., Biochem. 17:4900, (1978)) such as enolase, glyceraldehydes-3-phosphate dehydrogenase, hexokinase, pyruvatee decarboxylase, phosphofructokinase, glucose-6-phosphate isomerase, 3-phosphoglycerate mutase, pyruvate kinase, triosephosphate isomerase, phosphoglucose isomerase, and glucokinase. Other suitable vectors and promoters for use in yeast expression are further described in Fleer et al., Gene, 107:285-195 (1991). Other suitable promoters and vectors for yeast and yeast transformation protocols are well known in the art.

The term “marker sequence” or “marker gene” refers to a nucleic acid sequence capable of expressing an activity that allows either positive or negative selection for the presence or absence of the sequence within a host cell. For example, the P. pastoris URA5 gene is a marker gene because its presence can be selected for by the ability of cells containing the gene to grow in the absence of uracil (Nett et al., Yeast. 2003 November; 20(15):1279-90). Its presence can also be selected against by the inability of cells containing the gene to grow in the presence of 5-FOA. Marker sequences or genes do not necessarily need to display both positive and negative selectability. Non-limiting examples of marker sequences or genes from P. pastoris include ADE1, ARG4, HIS4 and URA3.

“Operatively linked” expression control sequences refers to a linkage in which the expression control sequence is contiguous with the gene of interest to control the gene of interest, as well as expression control sequences that act in trans or at a distance to control the gene of interest.

The term “expression control sequence” as used herein refers to polynucleotide sequences which are necessary to affect the expression of coding sequences to which they are operatively linked. Expression control sequences are sequences which control the transcription, post-transcriptional events and translation of nucleic acid sequences. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “recombinant host cell” (or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism.

The term “peptide” as used herein refers to a short polypeptide, e.g., one that is typically less than about 50 amino acids long and more typically less than about 30 amino acids long. The term as used herein encompasses analogs and mimetics that mimic structural and thus biological function.

The term “polypeptide” encompasses both naturally-occurring and non-naturally-occurring proteins, and fragments, mutants, derivatives and analogs thereof. A polypeptide may be monomeric or polymeric. Further, a polypeptide may comprise a number of different domains each of which has one or more distinct activities. The term “isolated protein” or “isolated polypeptide” is a protein or polypeptide that by virtue of its origin or source of derivation (1) is not associated with naturally associated components that accompany it in its native state, (2) exists in a purity not found in nature, where purity can be adjudged with respect to the presence of other cellular material (e.g., is free of other proteins from the same species) (3) is expressed by a cell from a different species, or (4) does not occur in nature (e.g., it is a fragment of a polypeptide found in nature or it includes amino acid analogs or derivatives not found in nature or linkages other than standard peptide bonds). Thus, a polypeptide that is chemically synthesized or synthesized in a cellular system different from the cell from which it naturally originates will be “isolated” from its naturally associated components. A polypeptide or protein may also be rendered substantially free of naturally associated components by isolation, using protein purification techniques well known in the art. As thus defined, “isolated” does not necessarily require that the protein, polypeptide, peptide or oligopeptide so described has been physically removed from its native environment.

A “modified derivative” refers to polypeptides or fragments thereof that are substantially homologous in primary structural sequence but which include, e.g., in vivo or in vitro chemical and biochemical modifications or which incorporate amino acids that are not found in the native polypeptide. Such modifications include, for example, acetylation, carboxylation, phosphorylation, glycosylation, ubiquitination, labeling, e.g., with radionuclides, and various enzymatic modifications, as will be readily appreciated by those skilled in the art. A variety of methods for labeling polypeptides and of substituents or labels useful for such purposes are well known in the art, and include radioactive isotopes such as ¹²⁵I, ³²P, ³⁵S, and ³H, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods for labeling polypeptides are well known in the art. See, e.g., Ausubel et al., Current Protocols in Molecular Biology, Greene Publishing Associates (1992, and Supplements to 2002) (hereby incorporated by reference).

The term “fusion protein” refers to a polypeptide comprising a polypeptide or fragment coupled to heterologous amino acid sequences. Fusion proteins are useful because they can be constructed to contain two or more desired functional elements from two or more different proteins. A fusion protein comprises at least 10 contiguous amino acids from a polypeptide of interest, more preferably at least 20 or 30 amino acids, even more preferably at least 40, 50 or 60 amino acids, yet more preferably at least 75, 100 or 125 amino acids. Fusions that include the entirety of the proteins of the present invention have particular utility. The heterologous polypeptide included within the fusion protein of the present invention is at least 6 amino acids in length, often at least 8 amino acids in length, and usefully at least 15, 20, and 25 amino acids in length. Fusions that include larger polypeptides, such as an IgG Fc region, and even entire proteins, such as the green fluorescent protein (“GFP”) chromophore-containing proteins, have particular utility. Fusion proteins can be produced recombinantly by constructing a nucleic acid sequence which encodes the polypeptide or a fragment thereof in frame with a nucleic acid sequence encoding a different protein or peptide and then expressing the fusion protein. Alternatively, a fusion protein can be produced chemically by crosslinking the polypeptide or a fragment thereof to another protein.

Amino acid substitutions can include those which: (1) reduce susceptibility to proteolysis, (2) reduce susceptibility to oxidation, (3) alter binding affinity for forming protein complexes, (4) alter binding affinity or enzymatic activity, and (5) confer or modify other physicochemical or functional properties of such analogs.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences.) In a preferred embodiment, a homologous protein is one that exhibits at least 65% sequence homology to the wild type protein, more preferred is at least 70% sequence homology. Even more preferred are homologous proteins that exhibit at least 75%, 80%, 85% or 90% sequence homology to the wild type protein. In a yet more preferred embodiment, a homologous protein exhibits at least 95%, 98%, 99% or 99.9% sequence identity. As used herein, homology between two regions of amino acid sequence (especially with respect to predicted structural similarities) is interpreted as implying similarity in function.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art. See, e.g., Pearson, 1994, Methods Mol. Biol. 24:307-31 and 25:365-89 (herein incorporated by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using a measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild-type protein and a mutein thereof. See, e.g., GCG Version 6.1.

A preferred algorithm when comparing a particular polypepitde sequence to a database containing a large number of sequences from different organisms is the computer program BLAST (Altschul et al., J. Mol. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The term “region” as used herein refers to a physically contiguous portion of the primary structure of a biomolecule. In the case of proteins, a region is defined by a contiguous portion of the amino acid sequence of that protein.

The term “domain” as used herein refers to a structure of a biomolecule that contributes to a known or suspected function of the biomolecule. Domains may be co-extensive with regions or portions thereof; domains may also include distinct, non-contiguous regions of a biomolecule. Examples of protein domains include, but are not limited to, an Ig domain, an extracellular domain, a transmembrane domain, and a cytoplasmic domain.

The terms “glucocerebrosidase,” “GCB,” “recombinant GCB,” or “recombinant GCB composition” and “rGCB” are used herein to mean any glucocerebrosidase produced from genetically manipulated glucocerebrosidase encoding nucleic acids, or any nucleotide sequence encoding β-glucocerebrosidase activity including human placental glucocerebrosidase.

As used herein, the term “N-glycan” refers to an N-linked oligosaccharide, e.g., one that is attached by an asparagines-N-acetylglucosamine linkage to an asparagine residue of a polypeptide. N-glycans have a common pentasaccharide core of Man₃GlcNAc₂ (“Man” refers to mannose; “Glc” refers to glucose; and “NAc” refers to N-acetyl; GlcNAc refers to N-acetylglucosamine). The term “trimannose core” used with respect to the N-glycan also refers to the structure Man₃GlcNAc₂ (“Man₃”), structurally defined as Manα1,3 (Manα1,6) Manβ1,4-GlcNAc β1,4-GlcNAc-Asn. It is also referred to as “paucimannose” structure.

A “high-mannose” type N-glycan described herein has more than eight mannose residues (e.g., Man₉GlcNAc₂—Man₁₂GlcNAc₂) on the GlcNAc₂ core structure (e.g., GlcNAc β1,4-GlcNAc-Asn).

Abbreviations used herein are of common usage in the art, see, e.g., abbreviations of sugars, above. Other common abbreviations include “PNGase”, which refers to peptide N-glycosidase F (EC 3.2.2.18); “GlcNAc Tr” or “GnT,” which refers to N-acetylglucosaminyltransferase enzymes; “NANA” refers to N-acetylneuraminic acid.

The term “occupancy” refers to an oligosaccharide moiety occupying an N-linked site on a glycoprotein. “Partial occupancy” refers to less than all N-linked sites occupied by a particular N-glycan structure, whereas “complete occupancy” refers to all N-linked sites occupied by a particular N-glycan structure. The glycosylation “occupancy” is generally determined by the ratio of each N-linked glycosylated polypeptides divided by total protein ratio of the proteins for each N-linked glycosylation site.

The term “predominant” or “predominantly” used with respect to the production of glycoproteins with one or more N-glycans, refers to an oligosaccharide structure which represents the major peak, as detected by matrix assisted laser desorption ionization time of flight mass spectrometry (MALDI-TOF) analysis. As used herein, the term “predominantly” or “the predominant” or “which is predominant” refers to N-glycan species that has the highest mole percent (%) of total N-glycans after the glycoprotein has been treated and released with PNGase and then analyzed by mass spectroscopy, (e.g., MALDI-TOF MS). In other words, the term “predominantly” refers to an individual entity (e.g, specific glycoform) which is present in greater mole percent than any other individual entity; or a combination of two or more specific glycoforms, each of which is present in greater mole percent than any other individual entity. For example, if a composition consists of species A in 40 mole percent, species B in 35 mole percent and species C in 20 mole percent and species D in 5 mole percent, the composition comprises predominantly species A. When the term “predominant” or “predominantly” is used with respect to two or more N-glycan species, those N-glycan species represent the two or more most “predominant” N-glycan species. In the above example, it may be stated that the predominant species of the glycoprotein composition is (1) species A; (2) a combination of A and B; (3) a combination of A, B and C; or (4) a combination of A, B, C and D. However, it cannot be stated that species A and C are the predominant species, because such combination leaves out species B, which is present in greater mole percent than species C. The terms “uniform glycosylation” or “uniformly glycosylated” used with respect to the production of N-glycans refers to a glycoprotein in which the oligosaccharide moiety that occupies the N-linked glycosylation sites on a glycoprotein comprises at least 60 mole % a single species of N-glycan; preferably at least 80 mole % a single species of N-glycan; and more preferably at least 90 mole % a single species of N-glycan, as detected by MALDI-TOF analysis.

The term “essentially free” used with respect to certain elements or moieties within a composition or compound will be understood to imply that the element or moiety is absent from the composition or compound, to such a degree that the element or moiety is present in mole percent or concentration of at least one order of magnitude lower than has previously been described, and preferably, at a mole percent or concentration that is below detectable measure, using currently available methods.

Throughout this specification and claims, the word “comprise” or variations such as “comprises” or “comprising”, will be understood to imply the inclusion of a stated integer or group of integers but not the exclusion of any other integer or group of integers.

As used herein, the term “molecule” means any compound, including, but not limited to, a small molecule, peptide, protein, sugar, nucleotide, nucleic acid, lipid, etc., and such a compound can be natural or synthetic.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used in the practice of the present invention and will be apparent to those of skill in the art. All publications and other references mentioned herein are incorporated by reference in their entirety. In case of conflict, the present specification, including definitions, will control. The materials, methods, and examples are illustrative only and not intended to be limiting.

Expression of Glucocerebrosidase in Lower Eukaryotic Host Cells

The present invention provides compositions and methods for expressing recombinant glucocerebrosidase including glucocerebrosidase compositions comprising terminal mannose residues on an N-linked glycan in lower eukaryotic host cells particularly in fungal hosts, such as yeast. The present invention, therefore, provides an isolated glycosylated GCB protein composition produced by the methods as disclosed herein.

In one aspect of the present invention, a method is provided for producing a composition of recombinant glucocerebrosidase [rGCB] protein comprising terminal mannose residues on an N-linked glycan in a lower eukaryotic host cell lacking at least one functional enzyme involved in hypermannosylation of proteins. Such methods comprise the step of (a) transforming the host cell with a nucleic acid sequence encoding an rGCB protein; and (b) culturing said lower eukaryotic host cell in conditions essentially free of neuraminidase or galactosidase suitable for expression of the rGCB protein to produce a composition of rGCB having predominantly an N-glycan glycoform selected from the group consisting of (a) Man₃GlcNAc₂, (b) Man₅GlcNAc₂, (c) Man₈GlcNAc₂ glycans, or a heterogenous pool of glycoforms (a) through (c). Reduction or elimination of hypermannosylation [i.e., production of high-mannose glycan structures] has been achieved by disrupting a gene encoding the initiating 1,6 mannosyltransferase activity (e.g, OCH1) in yeast, which produces glycoproteins comprising oligosaccharide moieties Man₅GlcNAc₂, Man₈GlcNAc₂ and high-mannose glycan structures (WO 02/00879, U.S. 20020137134, U.S. 20040018590 and Choi et al. Proc Natl Acad Sci USA. 2003 Apr. 29; 100(9):5022-7). Because high-mannose type glycans may be antigenic and undesirable for human therapeutics, the methods of the present invention provide for production of rGCB comprising predominantly glycans with fewer than nine mannose residues; and which are preferably essentially free of high-mannose residues.

The method may further comprise the expression of a mannosidase, such as a mannosidase having α-1,2-mannosidase activity, in a lower eukaryotic host cell. Expression of an α-1,2-mannosidase cleaves the Manα1,2 linkages on high-mannose glycan structures exposing terminal mannose residues (e.g., Manα1,3 or Manα1,6) on the rGCB glycoprotein. The method optionally provides for expression of at least one glycosylation enzyme selected from the group consisting of an N-acetylglucosaminyltransferases; UDP-N-acetylglucosamine transporter, a mannosidase II and β-hexosaminidase. Expression of these enzymes converts intermediate glycans suitable for in vivo and/or in vitro modification to produce a desired glycosylation structure on a rGCB. Accordingly, the present invention provides a method for producing a glycosylated rGCB composition using lower eukaryotic host cells.

In another aspect of the invention, methods are provided for producing a composition of rGCB protein in which high-mannose glycans comprise less than 30 mole %, preferably less than 20 mole %, and more preferably 10 mole % of total N-glycan structures.

In one embodiment, the present invention provides a method for producing a composition of rGCB protein comprising predominantly glycoforms (a) Man₃GlcNAc₂, (b) Man₅GlcNAc₂, and/or (c) Man₈GlcNAc₂. In preferred embodiments, these glycoforms (a) through (c) comprise at least 50 mole %, preferably 70 mole %; and more preferably at least 80 mole % of total N-glycan structures in the rGCB composition.

In another embodiment, the present invention provides a method for producing a composition of rGCB protein comprising at least 30 mole %, preferably 40 mole %, more preferably 50 mole % or more of a uniform N-glycan structure having fewer than 9 mannose residues. Preferred N-glycan structures are Man₃GlcNAc₂, Man₅GlcNAc₂, or Man₈GlcNAC₂.

In yet another embodiment, the present invention provides a method for producing a composition of recombinant glucocerebrosidase protein that lacks fucose. While recombinant glucocerebrosidase currently produced in mammalian cells comprise detectable amounts of fucose residues, the recombinant glucocerebrosidase produced in fungal hosts, such as yeast, by contrast, inherently lack the GDP-fucose pathway. In yet another embodiment, the present invention provides a method for producing a composition of recombinant glucocerebrosidase protein that lacks is essentially free of galactose. While rGCB currently produced in mammalian cells is then treated in vitro to enzymatically to remove much of the galactose residues, these enzymatic treatments are limited and the rGCB retains detectable amounts of galactose residues.

In a further embodiment, the present invention provides a method for producing a composition of recombinant glucocerebrosidase protein comprising at least 3, preferably 4 glycosylation sites. For instance, the recombinant glucocerebrosidase comprises N-linked sites at amino acid residues 19, 59, 146 and 270. Preferably, the method provides for glycosylation of at least 3 N-linked sites with uniform N-glycan structure, in which at least one site (e.g., AA 19) confers the lysosomal targeting necessary for proper targeting of the glucocerebrosidase enzyme. More preferably, all of the N-linked sites on the glucocerebrosidase are uniformly glycosylated with a single N-glycan structure having at least two terminal mannose residues (e.g., Man₃GlcNAc₂).

The method of the invention further includes a step of isolating an expressed GCB protein from the host. The method also provides the step for purifying glucocerebrosidase using chromatography and other known methods in the art.

An advantage of an isolated and purified polypeptide of the invention is that the recombinant glucocerebrosidase compositions are glycosylated with a desired glycosylation structure, free of high-mannose, galactose and/or fucose, and therefore, has reduced antigenicity and increased efficacy when administered as a therapeutic glycoprotein. Additionally, the purification step does not involve the removal of galactose or sialic acid residues. Furthermore, such GCB protein composition of the invention can have increased mannose receptor binding activities, thus providing GCB protein compositions that are more useful in therapeutic administration.

Fungal Hosts

In another aspect of the invention, the present invention provides a fungal host strain capable of recombinantly expressing an active glucocerebrosidase comprising a particular N-glycan structure (Example 1). In one embodiment, the fungal host strain is engineered to convert high-mannose type glycans to glycans having less than 9 mannose residues. Preferably, the fungal host is one that expresses at least one N-glycan selected from the group consisting of: GlcNAc₂Man₃GlcNAc₂, GlcNAcMan₃GlcNAc₂, Man₅GlcNAc₂, GlcNAcMan₅GlcNAc₂, Man₈GlcNAc₂ or Man₃GlcNAc₂. More preferably, the host cell is selected or engineered to produce a heterogenous pool of GCB protein with Man₃GlcNAc₂, Man₅GlcNAc₂ and Man₈GlcNAc₂; or a homogenous pool of GCB protein with Man₈GlcNAc₂, Man₅GlcNAc₂ or Man₃GlcNAc₂. Even more preferably, a host cell producing a pool of GCB protein with GlcNAc₂Man₃GlcNAc₂ is selected to produce Man₃GlcNAc₂ upon reaction with hexosaminidase either in vivo or in vitro.

In another embodiment, the host cells may lack mannosylphosphate transferase activity. Alternatively, the host has mannosylphosphate transferase activity. More preferably, the host cells lack sequences encoding one or more polypeptides having an enzymatic activity, e.g., an enzyme which affects O-glycan synthesis in a host such as protein mannosyltransferase (PMT) genes.

In yet another embodiment, the host cells also lack fucosyltransferase activity. Generally, host cells are selected that lack the GDP-Fucose biosynthetic pathway, however, host cells that have a GDP-Fucose pathway can be optionally engineered to lack fucose.

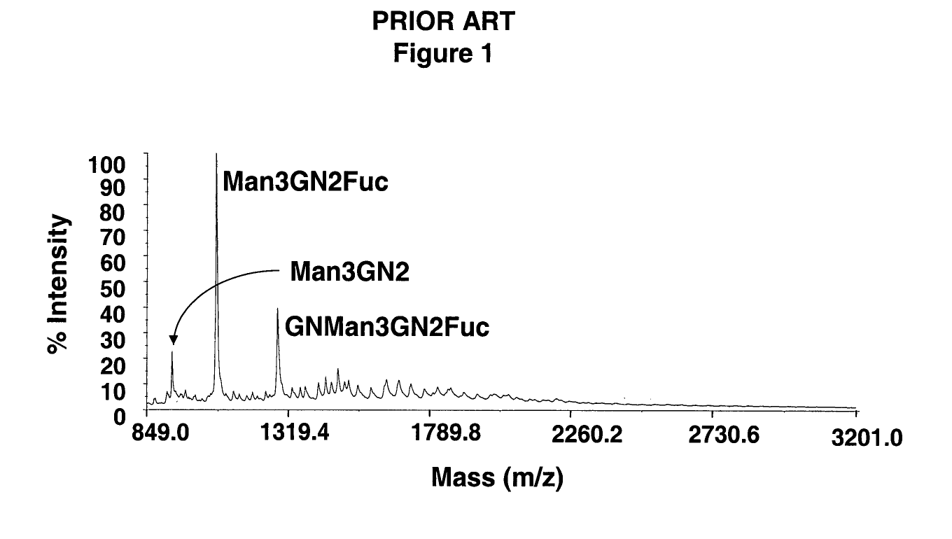

Following the methods provided herein, one skilled in the art can produce GCB protein with homogenous and heterogenous pools of terminal mannose glycans including Man₃GlcNAc₂ through Man₈GlcNAc₂ in a lower eukaryote. The host cells, therefore, produce either the GCB protein comprising the terminal mannose glycan structures or a recombinant GCB protein that can be subsequently modified with one enzymatic step in vitro (FIG. 1B).

A wide variety of suitable hosts exist for the production of recombinant glycosylated GCB of the present invention, however, preferred hosts for expressing glucocerebrosidase with terminal mannose structures include the following fungal hosts: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

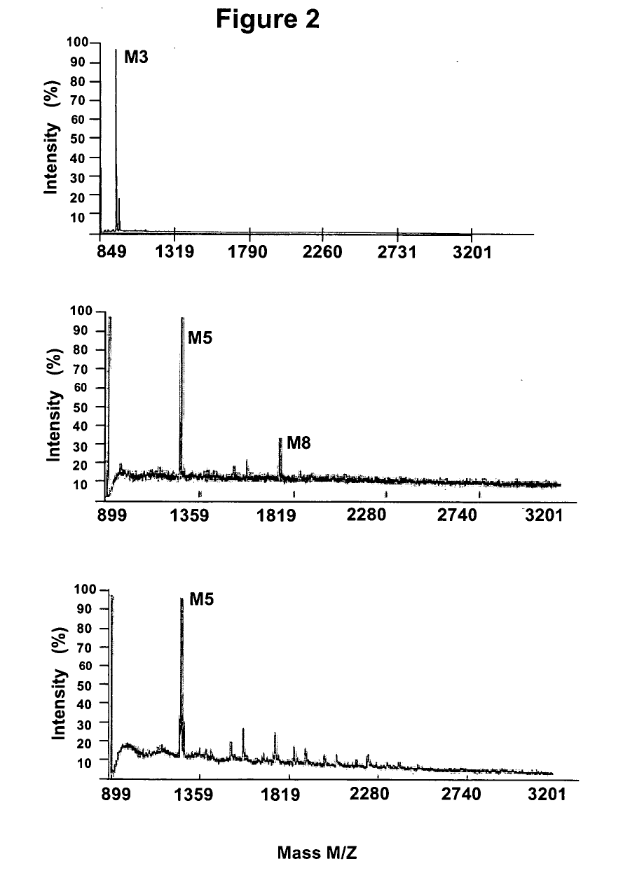

In one preferred embodiment, a yeast strain engineered to lack high-mannose type glycans expresses a gene encoding the human glucocerebrosidase. For instance, Glucocerebrosidase DNA (BC 003356) is cloned into a vector under a suitable promoter and transformed into various P. pastoris strains. The recombinant glucocerebrosidase gene is induced under a promoter and expressed and purified (Example 2). Preferably, the yeast strain has a P. pastoris YSH44 genetic background and expresses the gene encoding the human glucocerebrosidase. The yeast strain produces glucocerebrosidase comprising predominantly the GlcNAc₂Man₃GlcNAc₂ N-glycans (Hamilton et al., Science, 301, 1244-1246, 2003). The GCB protein from this strain is purified and treated in vitro with hexosaminidase resulting in a homogenous pool of glucocerebrosidase protein comprising predominantly Man₃GlcNAc₂ glycans with terminal mannose residues (FIG. 2A) (Example 2).

Alternatively, a gene encoding hexosaminidase is introduced into a yeast strain producing terminal GlcNAc residues on the N-glycan (e.g., P. pastoris YSH44). Preferably, the host expresses a Golgi-targeted hexosaminidase gene as described in Choi et al., 100, 5022-5027, 2003. The glucocerebrosidase protein purified from this strain has terminal mannose residues produced in vivo resulting in Man₃GlcNAc₂ glycans.

In another embodiment, glucocerebrosidase is expressed in a host that comprises a disruption in ALG3 (dolichyl-P-Man:Man₅GlcNAc₂-PP-dolichyl alpha-1,3 mannosyltransferase activity) and OCH1 genes, the host being free of high-mannose glycans with respect to the N-glycans. P. pastoris strain with this genetic background as described in WO 03/056914 produces glycoproteins comprising Manα1,2 Manα1,2 Manα1,3 (Manα1,6) Manβ1,4-GlcNAc β1,4-GlcNAc-Asn, herein after denoted, Man₅GlcNAc₂(B) glycans. Glucocerebrosidase protein isolated from this Δalg3 Δoch1 strain has Man₅GlcNAc₂(B) glycans. Transformation of the glucocerebrosidase gene in addition to the α-1,2 mannosidase I gene into this strain produces a glucocerebrosidase protein comprising predominantly Man₃GlcNAc₂ glycans.

In another embodiment the gene encoding glucocerebrosidase is expressed in a P. pastoris YJN168 genetic background (Choi et al, 2003). The host comprises a disruption in the OCH1 gene and expresses an α-1,2 mannosidase I enzyme with an MNS1 targeting sequence. The glycans from the glucocerebrosidase protein expressed from this strain exhibits Man₅GlcNAc₂ and Man₈GlcNAc₂ glycans (FIG. 2B). With the introduction of another α-1,2 mannosidase I enzyme targeted to the trans-Golgi as described in Choi et al, 2003, the expressed glucocerebrosidase protein comprises predominantly Man₅GlcNAc₂ glycans in vivo. Alternatively, this second α-1,2 mannosidase reaction can be carried out in vitro (Example 3).

In yet another embodiment the glucocerebrosidase gene is expressed in a P. pastoris YJN188 genetic background (Δoch1, +α1,2 MnsI/MNS1) (Choi et al, 2003) expressing an α-1,2 mannosidase I enzyme. This strain is similar to P. pastoris YJN168 (Δoch1, +α1,2 MnsI/MNN10), except the α-1,2 mannosidase enzyme is targeted to the Golgi with an MNN10 leader sequence. Targeting sequences to the ER or Golgi, catalytic domains of glycosidases, fusion enzymes and methods to target a particular glycosylation enzyme are described in WO 02/00879. According to this embodiment, the glycans from this strain are predominantly Man₅GlcNAc₂, with a more homogenous glycan pool than that of YJN168 (compare FIG. 2B with FIG. 2C). Thus, the number of mannose residues on the GCB protein can be controlled by using different targeting sequences as described in Choi et al., 2003 and in WO 02/00879.

In yet another preferred embodiment, the glucocerebrosidase gene is expressed in a P. pastoris BK64-1 genetic background (Choi et al., 2003). Glucocerebrosidase protein isolated from this strain comprises Man₈GlcNAc₂ glycans. The introduction of a Golgi-targeted α-1,2 mannosidase I enzyme into this strain as described in Choi et al., 2003 produces glucocerebrosidase protein with Man₅GlcNAc₂ glycans in vivo. Alternatively, this mannosidase reaction with Man₈GlcNAc₂ glycans can be carried out in vitro (Example 3).

Composition of Recombinant Glucocerebrosidase Protein

In one aspect, the present invention provides a composition of recombinant glucocerebrosidase protein comprising an N-glycan structure that comprises predominantly a glycoform selected from the group consisting of: (a) Man₃GlcNAc₂, (b) Man₅GlcNAc₂ or (c) Man₈GlcNAc₂ and a heterogenous pool of glycoforms (a) through (c). In one embodiment, the composition of recombinant glucocerebrosidase protein comprises less than 30 mole %, preferably less than 20 mole %, and more preferably less than 10 mole % N-glycoforms other than glycoforms (a) through (c).

In another embodiment, the present invention also provides a composition of recombinant glucocerebrosidase protein comprising an N-glycan structure that comprises a predominant glycoform selected from the group consisting of (a) Man₃GlcNAc₂, (b) Man₅GlcNAc₂, (c) Man₈GlcNAc₂ glycans, or a heterogenous pool of glycoforms (a) through (c), wherein the composition of recombinant glucocerebrosidase protein comprises at least 50 mole %, preferably 60 mole %,and more preferably 70 mole % or more of (a) Man₃GlcNAc₂, (b) Man₅GlcNAc₂ or (c) Man₈GlcNAc₂ glycan structures.

In yet another embodiment, the present invention also provides a composition of recombinant glucocerebrosidase protein comprising an N-glycan structure that comprises predominantly a glycoform selected from the group consisting of (a) Man₃GlcNAc₂, (b) Man₅GlcNAc₂, and/or (c) Man₈GlcNAc₂ glycans, wherein the composition of recombinant glucocerebrosidase protein comprises at least 30 mole %, preferably 40 mole %, more preferably 50 mole % or more of a particular N-glycan structure having fewer than 9 mannose residues on the core GlcNAc₂ of a glycoprotein.

In a further embodiment, the present invention provides a composition of recombinant glucocerebrosidase protein comprising less than 30, preferably 20, more preferably 10 mole % or less high-mannose glycans. In one embodiment, the GCB protein composition of the present invention is essentially free of N-glycans having mass over 1800 m/z. In an alternative embodiment, the GCB protein composition of the present invention is essentially free of N-glycans having mass over 1000 m/z.

In yet another embodiment, the present invention provides a composition of recombinant glucocerebrosidase protein that lacks fucose and also galactose residues on the glycan.

In a preferred aspect, the GCB composition is purified producing a GCB composition that is substantially free from substances that limit its effect or produce undesired side-effects.

It is contemplated that the GCB protein composition comprising at least 3 uniformly glycosylated N-glycan structures may have improved pharmacokinetics compared with that of placental-derived GCR or recombinant GCR (U.S. Pat. Nos. 5,236,838 and 5,549,892) and that the improved pharmacokinetics result at least in part from the improved affinity of the GCR for target cells compared with naturally occurring GCR or recombinant GCR.

Targeting of the Glucocerebrosidase Composition

The deficiency of glucocerebrosidase in Gaucher's patients leads to the accumulation of glucocerebroside glycolipids in the liver, spleen, bone marrow and lungs (Friedman, et al., 1999). However, only a small amount of the presently administered glucocerebrosidase (alglucerase) is effectively delivered to macrophages cells of these organs, and the distribution amongst these organs is not equal (Sato and Beutler, 1993, J. Clin. Invest., 91: 1909-1917; Bijsterbosch, et al., 1996, Eur. J. Biochem., 237: 344-349). Thus, a more efficiently targeted glucocerebrosidase enzyme is desired. Accordingly, in one embodiment, the present invention provides a composition of glucocerebrosidase protein comprising an N-glycan structure which confers an increase in the percent of administered glucocerebrosidase composition that reaches the liver, spleen, bone marrow and lungs. In another embodiment, the increase in targeted glucocerebrosidase composition is accomplished through optimization of the terminal mannose of the N-glycans. In yet another embodiment, the glucocerebrosidase protein is expressed in a host cell engineered to produce N-glycans of one predominant glycoform or one predominant set of glycoforms (e.g., Man₃GlcNAc₂, Man₅GlcNAc₂ and/or Man₈GlcNAc₂).

In another aspect, the GCB composition comprising a specific N-glycan conferring efficient targeting to macrophages of the liver, spleen, bone marrow and lungs is an isomer which has improved targeting over its cognate isomer. For example, the desired glycan is that produced in an Δalg3 mutant in yeast, resulting in a Man₅GlcNAc₂(B) glycan conferring improved targeting compared to the wild type Man₅GlcNAc₂ isoform. Thus, specific optimization of terminal mannose glycans for the most efficient GCB protein is also provided in the present invention.

Polypeptides Encoding Glucocerebrosidase Protein

In one aspect of the present invention, a human gene encoding β-glucocerebrosidase EC 3.2.1.45 is expressed in a lower eukaryotic host cell. A polynucleotide encoding human β-glucocerebrosidase is selected using various databases. The nucleotide sequence of the invention encoding a GCB polypeptide can be prepared by site-directed mutagenesis, synthesis or other methods known in the art. The encoded protein expressed in a lower eukaryotic host is properly folded and glycosylated.

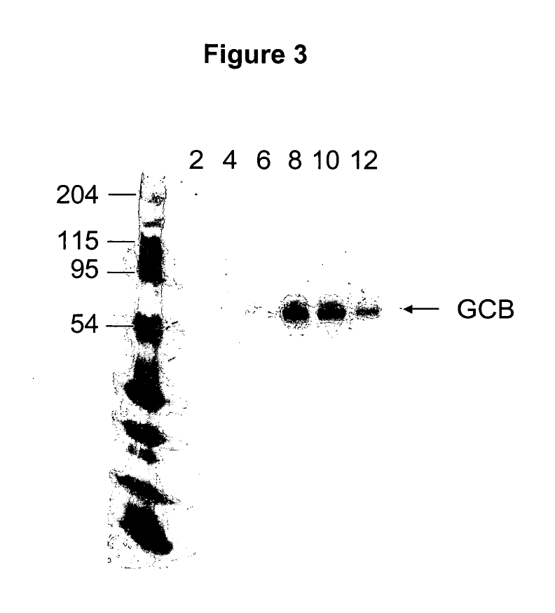

The nucleic acid encoding GCB is codon optimized (SEQ ID NO: 1). This may result in one or more changes in the primary amino acid sequence, such as a conservative amino acid substitution, addition, deletion or combination thereof. Non-conservative amino acid substitution may also result in functional GCB. FIG. 3 shows a Western blot of a codon optimized Glucocerebrosidase-His expressed from BK303 (Example 2).

The present invention also contemplates introduction of additional glycosylation site as described in U.S. patent application Ser. No. 2004/0009165. The additional glycosylation sites are preferably uniformly glycosylated.

In another aspect, to increase targeting to lysosomes, the mannose residues on the GCB composition is increased, however, the GCB composition has fewer than 9 mannose residues on the core GlcNAc₂ of an N-linked site on a glycoprotein.

The glycosylated GCB of the invention may further comprise a polymer molecule, for example, PEG, attached to the polypeptide. The PEGylated polypeptide is suitable for increasing serum half-life. The polypeptide according to this aspect is preferably a conjugated polypeptide comprising at least one non-oligosaccharide macromolecular moiety attached to N-terminus of the polypeptide.

Examples of suitable control sequences for use in yeast host cells include the promoters of the yeast α-mating system, the yeast triose phosphate isomerase (TPI) promoter, promoters from yeast glycolytic genes or alcohol dehydogenase genes (AOXI), the ADH2-4c promoter and the inducible GAL promoter.

The nucleotide sequence of the invention encoding a GCB polypeptide may or may not also include a nucleotide sequence that encodes a signal peptide. The signal peptide is generally used to secrete the polypeptide from the cells in which it is expressed. Such signal peptide, if present, should be one recognized by the cell chosen for expression of the polypeptide. The signal peptide may be homologous (e.g. be that normally associated with human GCB) or heterologous (i.e. originating from another source than human GCB) to the polypeptide or may be homologous or heterologous to the host cell, i.e. a signal peptide normally expressed from the host cell or one which is not normally expressed from the host cell. Examples include, HSA, PpKar2, invertase and Kilm1. Accordingly, the signal peptide may be prokaryotic, e.g. derived from a bacterium, or eukaryotic, e.g. derived from a mammalian, or insect, filamentous fungus or yeast cell.

The pharmaceutical GCB composition of the invention may be formulated in a variety of forms, including liquid, gel, lyophilized, or any other suitable form.

The present invention also provides pharmaceutical GCB compositions comprising adjuvants, a therapeutically effective amount of an agent or a pharmaceutically acceptable carrier. Compositions incorporating the GCB of the present invention may, therefore, include a pharmaceutical carrier and/or an adjuvant, generally non-toxic to recipients at the dosages and concentrations and is compatible with other ingredients of the formulation to provide a therapeutically convenient formulation and/or to enhance biochemical delivery and efficacy. The GCB of the present invention is formulated using known methods to formulate polypeptides. For example, the formulation can include mannitol, sodium citrates and polysorbate.

Various delivery systems are known and can be used to administer a compound of the invention. In one embodiment, the GCB composition is administered orally, by direct injection, by aerosol inhaler or by any suitable methods.

The following examples are for illustrative purposes and are not intended to limit the scope of the invention.

### EXAMPLES

### Example 1

Materials

Restriction and modification enzymes were from New England BioLabs. Oligonucleotides were obtained from the Dartmouth College Core facility (Hanover, N.H.) or Integrated DNA Technologies (Coralville, Iowa). The enzymes, peptide N-glycosidase F, mannosidases, and oligosaccharides were obtained from Glyko (San Rafael, Calif.). Metal chelating HisBind resin was from Novagen. Matrix-assisted laser desorption ionization.

### Example 2

Expression of Glucocerebrosidase in P. pastoris.

Glucocerebrosidase DNA (BC 003356) (SEQ ID NO: 1) (Tsuji et al., J Biol Chem 261, 50-53, 1986) is cloned into a pPICZA vector (Invitrogen) having the AOXI promoter and AOX1 terminal sequences. Using primers GBA/UP 5′AGCGCTAGACCATGTATTCCTAAGTCCTTCGGTT 3′ (SEQ ID NO:2) and GBA/LP 5′GGTACCTTATTGTCTGTGCCACAAGTAGGTGTGGAT 3′ (SEQ ID NO:3), GCB was subcloned into the multiple cloning site as an AfeI-KpnII fragment along with an upstream S. cerevisiae killer toxin signal sequence (EcoRI-AfeI fragment), which was codon optimized for P. pastoris, resulting in pBK376. The killer toxin signal sequence and the GCB gene were then excised as one EcoRI-KpnI fragment and cloned into a pPICZA-derived vector upstream of 3 glycine and 9 histidine sequences, resulting in pBK406. This pBK406 plasmid was transformed into various P. pastoris strains. Induction of the glucocerebrosidase gene is controlled by the methanol-inducible AOX1 promoter. After transformation of this vector, positive transformants are selected on Zeocin. GCB-His from pBK406 was expressed in strain BK303 (Δpno1Δmnn4 in YSH44 after kringle 3 protein is removed—U.S. patent application Ser. No. 11/020808). A Western blot of GCB-His expression from BK303 is shown in FIG. 3.

Specifically, for transformation of the glucocerebrosidase vector, the DNA is prepared by adding sodium acetate to a final concentration of 0.3 M. One hundred percent ice cold ethanol is then added to a final concentration of 70% to the DNA sample. The DNA is pelleted by centrifugation (12000g×10 min) and washed twice with 70% ice cold ethanol. The DNA is dried and resuspended in 50 μl of 10 mM Tris, pH 8.0. Yeast cultures to be transformed are prepared by expanding a yeast culture in BMGY (buffered minimal glycerol: 100 mM potassium phosphate, pH 6.0; 1.34% yeast nitrogen base; 4×10⁻⁵% biotin; 1% glycerol) to an O.D. of ˜2-6. The yeast cells are then made electrocompetent by washing 3 times in 1M sorbitol and resuspending in ˜1-2 mls 1M sorbitol. DNA (1-2 μg) is mixed with 100 μl of competent yeast and incubated on ice for 10 min. Yeast cells are then electroporated with a BTX Electrocell Manipulator 600 using the following parameters; 1.5 kV, 129 ohms, and 25 μF. One milliliter of YPDS (1% yeast extract, 2% peptone, 2% dextrose, 1M sorbitol) was added to the electroporated cells. Transformed yeast was subsequently plated on selective agar plates containing Zeocin.

### Example 3

Glucocerebrosidase Protein Isolation

A 10 ml culture of buffered glycerol-complex medium (BMGY) consisting of 1% yeast extract, 2% peptone, 100 mM potassium phosphate buffer (pH 6.0), 1.34% yeast nitrogen base, 4×10⁻⁵% biotin, and 1% glycerol was inoculated with a fresh colony of a P. pastoris strain transformed with glucocerebrosidase (e.g. YSH44, BK64-1, YSH44 or Δalg3Δoch1) and grown for 2 days. The culture was then transferred into 100 mls of fresh BMGY in a 1 liter flask for 1 day. This culture is then centrifuged and the cell pellet washed with BMMY (buffered minimal methanol: same as BMGY except 0.5% methanol instead of 1% glycerol). The cell pellet was resuspended in BMMY to a volume ⅕ of the original BMGY culture and placed in 1.5 liter fermentation reactor for 24 h. The secreted protein was harvested by pelleting the biomass by centrifugation and transferring the culture medium to a fresh tube. The collected supernatant His-tagged GCB was then purified on a Ni-affinity column, fractions were immunoblotted and glucocerebrosidase was digested with PNGase to release N-glycans (Choi et al., 2003).

Protein Purification

Glucocerebrosidase was purified from the collected BMMY supernatant medium by Ni-affinity chromatography using a Streamline Chelating resin from Amersham Biosciences. The column was charged with NiSO₄ then equilibrated with 20 mM Tris-HCl pH 7.9, 200 mM NaCl. The supernatant was applied directly to the column then washed with 4 volumes of the same buffer. Ten column volumes of an imidazol gradient (0-0.5M) in Tris buffer was then applied to the column. The fractions containing GCB were collected and submitted for Western and MALDI-TOF analysis.

Immunoblotting of Purified Glucocerebrosidase-His Protein

Even numbered fractions from the Ni-affinity column were collected and separated on a 4-20% gradient SDS-PAGE gel according to Laemmli, U. K. (1970) Nature 227, 680-685 and then electroblotted onto nitrocellulose membrane (Schleicher & Schuell). C-terminally His-tagged GCB was detected using an anti-His antibody (H-15) from Santa Cruz Biotech and an ECL kit (Amersham Pharmacia) FIG. 3.

Matrix Assisted Laser Desorption Ionization Time of Flight Mass Spectrometry

Molecular weights of the glycans were determined by using a Voyager DE PRO linear MALDI/TOF (Applied Biosciences) mass spectrometer with delayed extraction. The dried glycans from each well were dissolved in 15 μl of water, and 0.5 μl was spotted on stainless-steel sample plates and mixed with 0.5 μl of S-DHB matrix (9 mg/ml of dihydroxybenzoic acid/1 mg/ml of 5-methoxysalicylic acid in 1:1 water/acetonitrile/0.1% trifluoroacetic acid) and allowed to dry. Ions were generated by irradiation with a pulsed nitrogen laser (337 nm) with a 4-ns pulse time. The instrument was operated in the delayed extraction mode with a 125-ns delay and an accelerating voltage of 20 kV. The grid voltage was 93.00%, guide wire voltage was 0.1%, the internal pressure was <5×10⁻⁷ torr (1 torr=133 Pa), and the low mass gate was 875 Da. Spectrawere generated from the sum of 100-200 laser pulses and acquired with a 500-MHz digitizer. (Man)₅—(GlcNAc)₂ oligosaccharide was used as an external molecular weight standard. All spectra were generated with the instrument in the positive-ion mode.

### Example 4

Treatment of glucocerebrosidase-GlcNAc₂Man₅GlcNAc₂ with β-N-acetyl-hexosaminidase

The glycans are released and separated from the glucocerebrosidase protein by modification of a previously reported method (Papac, et al. A.J.S. (1998) Glycobiology 8, 445-454). After the proteins are reduced and carboxymethylated, and the membranes blocked, the wells are washed three times with water. The protein is deglycosylated by the addition of 30 μl of 10 mM NH₄HCo₃ pH 8,3 containing one milliunit of N-glycanase (Glyko, Novato, Calif.). After 16 hr at 37° C., the solution containing the glycans is removed by centrifugation and evaporated to dryness. The glycans are then dried in SC210A speed vac (Thermo Savant, Halbrook, N.Y.). The dried glycans are put in 50 mM NH₄Ac pH 5.0 at 37° C. overnight and 1 mU of hexos (Glyko, Novato, Calif.) is added.

### Example 5

Treatment of Glucocerebrosidase with α-1,2 mannosidase

Reconstitute the standard glycoprotein (20 μg) in 100 μl HPLC grade water. Aliquot 10 μl to a 0.6 ml siliconized tube. Evaporate the sample to dryness. Add 10 μl of 50 mM ammonium acetate. Add α-1,2 mannosidase from Trichoderma reseei (0.03 mU from R. Contreras Ghent, Belgium). Incubate the sample in enzyme for 16 to 24 hr at 37° C. Evaporate the sample to dryness. Reconstitute the sample in 10 μl of water. The sample is now ready for MALDI-TOF analysis.

1. A composition of recombinant glucocerebrosidase [rGCB] protein comprising an N-glycan structure that comprises predominantly a glycoform selected from the group consisting of: (a) Man₃GlcNAc₂, (b) Man₅GlcNAc₂, (c) Man₈GlcNAc₂ and a heterogenous pool of glycoforms (a) through (c), wherein less than 30 mole percent of N-glycans are other than glycoforms (a) through (c).

2. The composition of claim 1, wherein less than 20 mole percent of N-glycans are other than glycoforms (a) through (c).

3. The composition of claim 1, wherein less than 10 mole percent of N-glycans are other than glycoforms (a) through (c).

4. A composition of recombinant glucocerebrosidase [rGCB] protein comprising an N-glycan structure that comprises predominantly a glycoform selected from the group consisting of (a) Man₃GlcNAc₂, (b) Man₅GlcNAc₂, (c) Man₈GlcNAc₂ glycans, or a heterogenous pool of glycoforms (a) through (c), said composition of rGCB protein comprising at least 50 mole percent of glycoforms (a) through (c).

5. The composition of claim 4, wherein at least 60 mole percent of N-glycans one of glycoforms (a) through (c).

6. The composition of claim 4, wherein at least 70 mole percent of N-glycans one of glycoforms (a) through (c).

7. A composition of recombinant glucocerebrosidase [rGCB] protein comprising an N-glycan structure that comprises predominantly a glycoform selected from the group consisting of (a) Man₃GlcNAc₂, (b) Man₅GlcNAc₂, and/or (c) Man₈GlcNAc₂ glycans, wherein said composition of rGCB protein comprises at least 30 mole percent of a predominant N-glycan structure having fewer than 9 mannose residues.

8. The composition of claim 7, wherein said composition comprises at least 40 mole percent of a predominant N-glycan structure having fewer than 9 mannose residues.

9. The composition of claim 7, wherein said composition comprises at least 50 mole percent of a predominant N-glycan structure having fewer than 9 mannose residues.

10. The composition of claims 1, 4, or 7, wherein said composition of rGCB protein comprises less than 30 mole percent high-mannose glycans.

11. The composition of claim 10, wherein said composition of rGCB protein comprises less than 20 mole percent high-mannose glycans.

12. The composition of claim 10, wherein said composition of rGCB protein comprises less than 10 mole percent high-mannose glycans.

13. The composition of claim 1, 4 or 7, wherein said composition of rGCB protein is essentially free of fucose and/or galactose residues.

14. A method for producing a composition of recombinant glucocerebrosidase [rGCB] protein in a lower eukaryotic host cell, said lower eukaryotic host cell lacking at least one functional enzyme involved in hypermannosylation of proteins, said method comprising:
a. transforming said lower eukaryotic host cell with a recombinant nucleotide sequence encoding an rGCB protein; and
b. culturing said lower eukaryotic host cell in conditions essentially free of neuraminidase or galactosidase suitable for expression of the rGCB protein to produce predominantly a glycoform selected from the group consisting of (a) Man₃GlcNAc₂, (b) Man₅GlcNAc₂, (c) Man₈GlcNAc₂ glycans, or a heterogenous pool of glycoforms (a) through (c).

15. The method of claim 14, wherein the lower eukaryotic host cell lacks at least one functional enzyme involved in hypermannosylation selected from the group consisting of: the ALG3 gene; and the OCH1 gene.

16. The method of claim 14, wherein the lower eukaryotic host cell expresses at least one exogenous gene selected from the group consisting of mannosidases; mannosyltransferases; N-acetylglucosaminyltransferases; UDP-N-acetylglucosamine transporters; and phosphomannosyltransferases.

17. The method of claim 14, wherein the lower eukaryotic cell is selected from the following species: Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha, Kluyveromyces sp., Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum and Neurospora crassa.

18. The method of claim 14, wherein less than 30 mole percent of the N-glycans in said rGCB protein composition comprises high-mannose glycans.

19. The method of claim 14, wherein less than 20 mole percent of the N-glycans in said rGCB protein composition comprises high-mannose glycans.

20. The method of claim 14, wherein less than 10 mole percent of the N-glycans in said rGCB protein composition comprises high-mannose glycans.

21. The method of claim 14, wherein less than 30 mole percent of the N-glycans in said rGCB protein composition comprises a glycan other than (a) Man₃GlcNAc₂, (b) Man₅GlcNAc₂, or (c) Man₈GlcNAc₂.

22. The method of claim 14, less than 20 mole percent of the N-glycans in said rGCB protein composition comprises a glycan other than (a) Man₃GlcNAc₂, (b) Man₅GlcNAc₂, or (c) Man₈GlcNAc₂.

23. The method of claim 14, wherein less than 10 mole percent of the N-glycans in said rGCB protein composition comprises a glycan other than (a) Man₃GlcNAc₂, (b) Man₅GlcNAc₂, or (c) Man₈GlcNAc₂.

24. The method of claim 14, wherein at least 50 mole percent of the N-glycans in said rGCB protein composition comprises Man₃GlcNAc₂, Man₅GlcNAc₂, or Man₈GlcNAc₂.

25. The method of claim 14, wherein at least 60 mole percent of the N-glycans in said rGCB protein composition comprises Man₃GlcNAc₂, Man₅GlcNAc₂, or Man₈GlcNAc₂.

26. The method of claim 14, wherein at least 70 mole percent of the N-glycans in said rGCB protein composition comprises Man₃GlcNAc₂, Man₅GlcNAc₂, or Man₈GlcNAc₂.

27. The method of claim 14, wherein at least 30 mole percent of the N-glycans in said rGCB protein composition comprises a predominant N-glycan structure having fewer than 9 mannose residues.

28. The method of claim 14, wherein at least 40 mole percent of the N-glycans in said rGCB protein composition comprises a predominant N-glycan structure having fewer than 9 mannose residues.

29. The method of claim 14, wherein at least 50 mole percent of the N-glycans in said rGCB protein composition comprises a predominant N-glycan structure having fewer than 9 mannose residues.

30. The method of claim 14, wherein said composition of rGCB protein is essentially free of fucose and/or galactose.

31. A method of treating Gaucher's type disease comprising administration of a therapeutically effective amount of a recombinant glucocerebrosidase [rGCB] composition, said rGCB comprising predominantly N-glycan structures selected from the group consisting of (a) Man₃GlcNAc₂, (b) Man₅GlcNAc₂, (c) Man₈GlcNAc₂ glycoforms, and a heterogenous pool of glycoforms (a) through (c), said composition of rGCB protein comprising at least 30 mole percent of glycoforms (a) through (c).

32. The method of claim 31, wherein at least 40 mole percent of the N-glycan structures comprises glycoforms (a) through (c).

33. The method of claim 31, wherein at least 50 mole percent of the N-glycan structures comprises glycoforms (a) through (c).

34. The method of claim 31, wherein the rGCB composition further comprises a pharmaceutically acceptable carrier.

35. A recombinant glucocerebrosidase [rGCB] protein composition comprising occupancy of at least 3 N-linked sites with a predominant glycoform selected from the group consisting of (a) Man₃GlcNAc₂, (b) Man₅GlcNAc₂ and (c) Man₈GlcNAc₂ glycans for at least 50 mole percent of the rGCB protein composition.

36. The GCB protein composition of claim 35 wherein the N-glycans are essentially free of N-glycans having mass over 1000 m/z.

37. The GCB protein composition of claim 35 wherein the N-glycans are essentially free of N-glycans having mass over 1800 m/z.
